(19) United States
(12) Patent Application Publication
(10) Pub. No.: US 2020/0375890 A1
(43) Pub. Date: Dec. 3, 2020

(54) IMPLANTABLE AND REMOVABLE DRUG DELIVERY DEVICE

(71) Applicant: Particle Sciences, Inc., Bethlehem, PA (US)

(72) Inventors: Mark A. Mitchnick, Bethlehem, PA (US); Kimberly A. Zubris, Bethlehem, PA (US); Brett Braker, Bethlehem, PA (US)

(21) Appl. No.: 16/496,522

(22) PCT Filed: Mar. 16, 2018
(86) PCT No.: PCT/US2018/022874

§ 371 Date: Sep. 23, 2019

Related U.S. Application Data
(60) Provisional application No. 62/475,407, filed on Mar. 23, 2017.

(51) Int. Cl. A61K 9/00; A61K 45/06

(52) U.S. Cl. CPC A61K 9/0024; A61K 45/06

(57) ABSTRACT

The disclosed technology provides an improved drug delivery device which is easily implantable and removable, as well as method of using and making the same. The described drug delivery device includes: (a) a wall comprising at least in part a polymeric composition, which surrounds and forms; (b) a compartment; (c) a drug composition in the compartment which includes an active agent; (d) at least one passageway in the wall for releasing the active agent from said drug composition to said environment of use. The device (i) can be implanted in an environment of use, (ii) is able to maintain its structural integrity for a period of use to allow for the release of the active agent, and (iii) can then be removed from the environment of use.

### FIELD OF THE INVENTION

There is provided herein an improved drug delivery device which is easily implantable and removable, as well as method of using and making the same.

### BACKGROUND

Implantable medical devices are often used to treat subjects, including patients, and such devices can be used in a variety of locations within the anatomy of the subject. Such devices can be permanently or semi-permanently implanted in the anatomy to provide treatment to the subject, including a patient.

Implantable medical devices may be used to deliver a therapeutic agent in the vicinity of the implant, thereby providing localized as opposed to systemic delivery. Such devices can be described as drug-eluting devices. For example, in the case of localized vascular disease, systemic administration of a drug may not be desirable because the drug may have unwanted effects on parts of the body which are not to be treated, or because treatment of the diseased vasculature requires a high concentration of drug that may not be achievable by systemic administration. It is therefore often desirable to administer drugs in a localized manner to vascular tissues.

Various devices for localized drug delivery are known, including stents or stent-grafts which have been coated with an elutable drug.

However, many of these devices included complicated features that may not function properly over the entire use of the device. In other instances, the device may not remain intact over the course of its use and begin to break down or deteriorate before its period of use is complete. Often, it is advantageous and even necessary to remove the device from the subject after the period of use. When the device is susceptible to premature deterioration it can lead to complications in the subject.

There is a need for an improved drug delivery device which is easily implantable and removable from a subject. There is a need for a drug delivery device that remains intact during the entire period it is put to use and that remains structurally sound so that it may be easily removed in one piece. There is also a need for improved means of maintaining and/or controlling the release rate of drug from such devices to the subject.

### SUMMARY

The disclosed technology provides improved drug delivery devices that are implantable into a patient and/or environment of use, and which can then be easily removed. The disclosed technology also provides drug delivery devices with improved means for maintaining and/or controlling the release of drugs to the patient and/or environment of use.

The disclosed technology provides a drug delivery device for administering a therapeutically effective amount of an active agent to an environment of use, where the device includes: (a) a wall comprising at least in part a polymeric composition, a metallic material, or a combination thereof; which wall surrounds and forms; (b) a compartment; (c) a drug composition in the compartment, where the drug composition includes the active agent, wherein said drug composition is in a formed shape that fits within said compartment; and (d) at least one passageway in the wall communicating with the compartment and the exterior of the device for releasing the active agent from said drug composition to said environment of use; and where said device (i) can be implanted in the environment of use, (ii) is able to maintain its structural integrity in the environment of use for a period of use such that a therapeutically effective amount of the active agent releases from said drug composition into said environment of use, and (iii) can then be removed from the environment of use. In some embodiments, the composition that forms the device will degrade but at a rate slower than the drug release rate, such that all or substantially all of the drug will be released before the device loses structural integrity.

The disclosed technology further provides the described drug delivery device where each passageway is of a shape or size that, at least initially if not for the entire period of use, prevents said drug composition from passing through the passageway and out of the compartment.

The disclosed technology further provides the described drug delivery device where the release rate of the active agent from said drug composition to said environment of use is not controlled by the permeability of the polymeric material by the active agent or by said any fluids present in the environment of use. That is, the drug delivery rate is primarily controlled by the dissolution of the active agent into fluids present in the environment of use that come into contact with the drug composition.

The disclosed technology further provides for the described drug delivery device where the period of use is at least 1 month, or at least 3 months, or at least 6 months, or even a period of time needed to release substantially all of the active agent in the environment of use.

The disclosed technology further provides for the described drug delivery device where the polymeric material includes: (a) a biodegradable polymer; (b) a non-biodegradable polymer; (c) any combination thereof. In some of these embodiments, the device is free of metallic materials.

The disclosed technology further provides for the described drug delivery device where the active agent includes one or more analgesics, anti-anginal agents, anti-arrhythmic agents, anti-angiogenic agents, antibacterial agents, anti-benign prostate hypertrophy agents, anti-coagulants, anti-depressants, anti-diabetic agents, anti-epileptic agents, anti-fungal agents, anti-gout agents, anti-hypertensive agents, anti-inflammatory agents, anti-malarial agents, anti-migraine agents, anti-muscarinic agents, anti-neoplastic agents, anti-obesity agents, anti-osteoporosis agents, anti-parkinsonian agents, anti-protozoal agents, anti-thyroid agents, anti-urinary incontinence agents, anti-viral agents, anxiolytics, beta-blockers, cardiac inotropic agents, cognition enhancers, corticosteroids, COX-2 inhibitors, diuretics, erectile dysfunction improvement agents, essential fatty acids, gastrointestinal agents, histamine receptor antagonists, hormones, immunosuppressants, keratolyptics, leukotriene antagonists, lipid regulating agents, macrolides, muscle relaxants, non-essential fatty acids, nutritional agents, nutritional oils, protease inhibitors, stimulants, or any combination thereof.

The disclosed technology further provides for the described drug delivery device where the environment of use is the mouth, ear canals, skull, gastrointestinal tract, wounds, teeth cavities, vagina, anus, stoma, eye cavities, kidneys, testicles, prostate, lungs, transplanted organs, blood vessels, the biliary tract, the urinary tract, the intestinal tract, nasal cavity, neural sheath, intervertebral regions, bone cavities, esophagus, intrauterine spaces, pancreatic and bile ducts, rectum, any location within the body just beneath an outer surface of skin, any previously intervened body spaces that have implants present, or any combination thereof.

The disclosed technology further provides the described drug delivery device where the device has a series of passageways arranged in a pattern on at least one surface of said device.

The disclosed technology further provides for the described delivery device where the device is made by means of 3D printing, and where the device is printed as a single continuous piece where said drug composition is inserted into the compartment within the device while the device is being printed. The device is then completed with the drug composition in place inside the device.

The disclosed technology further provides for the described drug delivery device where the device includes a mesh material positioned on the wall of the device such that it covers one or more of the passageways present in the wall of the device.

The disclosed technology further provides for the described drug delivery device where the mesh material covers all of the passageways of the device. In some embodiments, the mesh material is made of a polymeric material that swells when in contact with an environmental agent that may be present in the environment of use, such that when the environmental agent is present, the mesh material swells and substantially blocks the passageways of said device, restricting the release of the active agent from the drug composition to the environment of use.

The disclosed technology further provides for the described drug delivery device where the mesh material comprises a biodegradable polymer. In such embodiments, all of the passageways may be covered by the mesh material and as the mesh material biodegrades the passageways may become more open and/or available to allow fluids present in the environment of use to pass into and out of the device, thus increasing the release rate of the active agent into the fluids and so also into the environment of use.

The disclosed technology further provides for the described drug delivery device where the mesh material is made of a cross-linked polyacrylic acid polymer that is a carbomer homopolymer, a carbomer copolymer, a carbomer interpolymer, a polycarbophil or a mixture thereof.

The disclosed technology further provides where the drug delivery device is coated, treated, or both with a material to minimize scaring or encapsulation of said device by the environment of use.

The disclosed technology further provides for the described drug delivery device where the device: (i) is not an osmotic device; (ii) is not bioresorbable; (iii) is not biodegradable; (iv) does not contain a semi-permeable membrane; (v) is not in the shape of a ring; (vi) is not significantly permeable to the active agent, any fluids present in the environment of use, or both; or (vii) all of the above.

The disclosed technology further provides a method for using the described drug delivery device to administer a therapeutically effective amount of an active agent to an environment of use, where the drug delivery device may be any of the devices described herein, and where the method includes the steps of: (I) implanting a drug delivery device into an environment of use; (II) administering a therapeutically effective amount of an active agent from said drug composition to said environment of use during a period of use; and (III) removing said drug delivery device from said environment of use; where the drug delivery device maintains its structural integrity during said period of use.

### DETAILED DESCRIPTION

Various preferred features and embodiments will be described below by way of non-limiting illustration.

The disclosed technology provides improved drug delivery devices that may be implanted into a patient and/or environment of use for the purpose of delivery one or more active agents to the patient and/or environment of use, where the drug delivery device may then be easily removed from the patient and/or environment of use after the active agent has been delivered. Alternatively, the device may be left in to degrade over a time period that exceeds the drug delivery time (such that the device remains fully intact for the duration of time where drug delivery occurs).

The Drug Delivery Device

The disclosed technology provides a drug delivery device for administering a therapeutically effective amount of an active agent to an environment of use, where the device includes: (a) a wall comprising at least in part a polymeric composition, a metallic material, or a combination thereof; which wall surrounds and forms; (b) a compartment; (c) a drug composition in the compartment, where the drug composition includes the active agent, wherein said drug composition is in a formed shape that fits within said compartment; and (d) at least one passageway in the wall communicating with the compartment and the exterior of the device for releasing the active agent from said drug composition to said environment of use; and where said device (i) can be implanted in the environment of use, (ii) is able to maintain its structural integrity in the environment of use for a period of use such that a therapeutically effective amount of the active agent releases from said drug composition into said environment of use, and (iii) can then, optionally, be removed from the environment of use. In some embodiments, the device is removed after the release is complete. In other embodiments, the device is left in the environment of use to degrade slowly over time, but only after the drug release is complete.

The shape and form of the device is not overly limited. In some embodiments, the device is in the shape of a small box with a hollow compartment inside that can accommodate the drug compositions. The device can be approximately rectangular in shape, or it can be approximately in the shape of a disc, or it can be approximately the shape of a cube, or it can be in the shape of a rod or tube, or it can be in the shape of a sphere. The device can be made for a specific space where the device contours to the space. In still other embodiments, the device is in the shape of a ring where the ring is made up of a hollow tube. In such embodiment, the hollow area of the tube serves as the compartment to hold the drug composition and one or more openings and passageways may be in the ring. In each of these embodiments, the device has a hollow compartment to accommodate the drug composition.

The disclosed technology further provides the described drug delivery device where each passageway is of a shape or size that prevents solid or undissolved said drug composition from passing through the passageway and out of the compartment.

The shape of the passageway is not overly limited, so long as the passageway is small enough to prevent the entire drug composition to pass through the passageway, and out of the device. In some embodiments, the passageways are small enough to prevent the entire drug composition to pass through the passageway and out of the device during the period of use.

In one embodiment, the device includes one passageway. In one embodiment, the device includes more than one passageway. In one embodiment, the device includes two passageways, or two or more passageways. In one embodiment, the device includes three passageways, or three or more passageways. In one embodiment, the device includes four passageways, or four or more passageways. In one embodiment, the device includes at least six passageways. In one embodiment, the device includes at least ten passageways.

In one embodiment, the device has one or more passageways where each passageway is covered by a mesh, where the openings in the mesh are small enough to prevent the entire drug composition to pass through the passageway. In such embodiments, the passageways on the device covered by the mesh can be large enough for the drug composition to enter the device. In such embodiments, the passageway can be used to insert the drug composition into the device, and then when the mesh is placed over the passageway and fastened on to the device, the mesh becomes part of the device, and the only passageways in the device are the openings in the mesh.

The disclosed technology further provides the described drug delivery device where the device has a series of passageways arranged in a pattern on at least one surface of said device.

The pattern of the passageways are not overly limited. In some embodiments, the passageways are arranged in the pattern of a circle and/or oval (that is, if you were to draw a line through each of the passageways on the surface of the device, the line would form the shape of a circle and/or oval). In some embodiments, the passageways are in the pattern of a series of concentric circles and/or ovals, where the layout of the concentric circles and/or ovals used to place the passageways and the size of the passageways are such that the passageways remain separated from each other.

In some embodiments, the passageways are arranged in the pattern of a square, rectangle and/or triangle (that is, if you were to draw a line through each of the passageways on the surface of the device, the line would form the shape of a square, rectangle and/or triangle). In some embodiments, the passageways are in the pattern of a series of concentric squares, rectangles and/or triangles.

In some embodiments, the passageways of the device are only present on side and/or face of the device (e.g., if the device is in the shape of rectangular box, the passageways are only present on one face of the device). In some embodiments, the passageways of the device are present on opposite sides and/or faces of the device (e.g., if the device is in the shape of rectangular box, the passageways are present on opposite faces of the device). In some embodiments, the passageways of the device are present on each side and/or face of the device (e.g., if the device is in the shape of rectangular box, the passageways are present on each face of the device).

The disclosed technology further provides for the described delivery device where the device is made by means of 3D printing, and where the device is printed as a single continuous piece where said drug composition is inserted into the compartment within the device while the device is being printed. The device is then completed with the drug composition in place inside the device.

The means or type of 3D printing useful in practicing the disclosed technology is not overly limited. In some embodiments, fused deposition modeling (FDM) 3D printing is used. In some embodiments, selective laser sintering (SLS) 3D printing is used. In some embodiments, digital light processing (DLP) 3D printing is used. In some embodiments, stereolithography (SLA) 3D printing is used. In some embodiments, selective laser melting (SLM) 3D printing is used. In some embodiments, electronic beam melting (EBM) 3D printing is used. In some embodiments, laminated object manufacturing (LOM) (SLM) 3D printing is used.

The device can be printed as a single piece by means of 3D printing or other similar forms of manufacturing. In such embodiments, the drug composition may be inserted into the compartment of the device when the device is partially formed, allowing access to the compartment before the entire device is in place. Thus, a drug composition in the form of a tablet or pill, where the shape and size of the tablet or pill would prevent the drug composition from passing through the openings in the finished device can be easily inserted into the device during its manufacture. Once the drug composition is in place, the manufacturing of the device can be completed around the drug composition, enclosing the drug composition in the described compartment. In other embodiments, the 3D printer may be equipped to print the drug composition as well as the device, allowing for the device to be printed in a single process.

Where the device is not 3D printed, the device may be made up of two or more pieces such that the device is assembled in such a way that the drug composition assembled with the pieces of the device and the parts of the device are fastened together around the drug composition forming the device with the drug composition enclosed in the compartment within the device.

In still other embodiments, the device may be made from a single piece where, or even from two or more pieces that are assembled, where the drug composition is in the form of a liquid, gel, paste, melt, slurry, or other non-solid or semi solid form. In such embodiments, the drug composition may be added to the device through the passageways in the fully assembled device. Means of addition include but are not limited injection, pouring, and the like. In some embodiments, the drug composition is injected into the device. Once the drug composition, in its non-solid state, is in position within the device, the drug composition may then solidify, thicken, crosslink, coalesce or otherwise become a solid, semi-solid, or more solid like substance such that it can no longer pass through the openings and/or passageways in the device. The conversion and/or transition of the drug composition may be assisted by changes in temperature, for example, a cure step, dehydration and/or evaporation of liquid in the drug composition, or various other techniques.

The disclosed technology further provides for the described drug delivery device where the device includes a mesh material positioned on the wall of the device such that it covers one or more of the passageways present in the wall of the device.

The disclosed technology further provides for the described drug delivery device where the mesh material covers all of the passageways of the device. In some embodiments, the mesh material is made of a polymeric material that swells when in contact with an environmental agent that may be present in the environment of use, such that when the environmental agent is present, the mesh material swells and substantially blocks the passageways of said device, restricting the release of the active agent from the drug composition to the environment of use.

In such embodiments, the openings on the mesh become the openings and passageways of the device. The mesh can be attached and/or fastened to the device in numerous ways. In some embodiments, the mesh is glued to the device. In some embodiments, the mesh is melt bonded to the device. In some embodiments, the mesh is printed onto the device by a 3D printer using any of the techniques described above.

The polymeric composition useful in the disclosed technology is not overly limited. In some embodiments, the polymeric material includes: (a) a biodegradable polymer; (b) a non-biodegradable polymer; (c) any combination thereof.

In some embodiments, the device is made from a polymeric composition that includes a biodegradable polymer. Suitable biodegradable polymers include poly(lactic acid), polyhydroxyalkanoate, polycaprolactone, aliphatic copolyesters made from alkene diols and aliphatic dicarboxylic acids, aromatic copolyesters made from alkene diols and aromatic dicarboxylic acids, or any combination thereof.

In some embodiments, the device is made from a polymeric composition that includes a non-biodegradable polymer. Suitable non-biodegradable polymers include poly(ethylene) (PE), poly(propylene) (PP), poly(tetrafluroethylene) (PTFE), poly(methylmethacrylate), ethylene-co-vinylacetate (EVA), poly(dimethylsiloxane) (PDMS), poly(ether-urethanes) (PU), poly(ethylene terephthalate) (PET), poly(sulphone), poly(ethyleneoxide), or any combination thereof.

In some embodiments, the polymeric material is a mixture of materials which include one or more of any of the materials above, and one of more additional polymers, including but not limited to poly(lactide-co-glycolide) (PLGA), polylactide (PLA), polyglycolide (PGA), D-lactide, D,L-lactide, L-lactide, D,L-lactide-co-ϵ-caprolactone, L-lactide-co-ϵ-caprolactone, D,L-lactide-co-glycolide-co-ϵ-caprolactone, poly (D,L-lactide-co-caprolactone), poly (L-lactide-co-caprolactone), poly (D-lactide-co-caprolactone), poly(D,L-lactide), poly(D-lactide), poly(L-lactide), polyesteramide, or a combination thereof. In some embodiments, the polymeric material is made up of one or more of these polymers.

The metallic material useful in the disclosed technology is not overly limited. In some embodiments, the metallic material includes stainless steel, cobalt-chrome, titanium, nickel-titanium, or any combination thereof. In some embodiments, the metallic material includes: 316L stainless steel; and/or MP35N, L-605, and/or Elgiloy cobalt-chrome; Ti-6AI-4V and/or CP titanium; and/or nitinol nickel-titanium. In some embodiments, the wall (and even the entire medical device) is made of a polymeric material and is free of metallic materials.

The disclosed technology further provides that the device may be coated or treated in such a way to minimize scaring or encapsulation of the device by the body.

The Drug Composition

The disclosed technology includes a drug composition. The drug composition includes an active agent for administration to the patient, subject, and/or environment of use.

The drug composition suitable for use in the devices disclosed herein is not overly limited. The drug composition is in a form and/or shape that allows it to occupy the compartment within the device. In some embodiments, the drug composition is in the form of a table or pill that fits within the compartment within the device.

The active agent for use in the drug composition is not overly limited and may contain one more active agents for various purposes. The drug composition may also include one or more additives that are typical for use in such compositions.

Suitable additional additives that may be present in the drug composition include binders, fillers and disintegrants, for example, starch. As noted above, the drug composition may take various forms, which include, but are not limited to, liquids, soft substances, powder-like substances, and hard pharmaceutical substances such as soft capsules, hard capsules and tablets. In one embodiment, the pharmaceutical dosage form is a capsule. In another embodiment, the capsule can be coated with a pH-sensitive coating. The pH-sensitive coating may prevent dissolution until the drug composition comes into contact with fluids in the desired environment of use, thus making sure drug release does not occur until that point.

The disclosed technology further provides for the described drug delivery device where the active agent includes one or more analgesics, anti-anginal agents, anti-arrhythmic agents, anti-angiogenic agents, antibacterial agents, anti-benign prostate hypertrophy agents, anti-coagulants, anti-depressants, anti-diabetic agents, anti-epileptic agents, anti-fungal agents, anti-gout agents, anti-hypertensive agents, anti-inflammatory agents, anti-malarial agents, anti-migraine agents, anti-muscarinic agents, anti-neoplastic agents, anti-obesity agents, anti-osteoporosis agents, anti-parkinsonian agents, anti-protozoal agents, anti-thyroid agents, anti-urinary incontinence agents, anti-viral agents, anxiolytics, beta-blockers, cardiac inotropic agents, cognition enhancers, corticosteroids, COX-2 inhibitors, diuretics, erectile dysfunction improvement agents, essential fatty acids, gastrointestinal agents, histamine receptor antagonists, hormones, immunosuppressants, keratolyptics, leukotriene antagonists, lipid regulating agents, macrolides, muscle relaxants, non-essential fatty acids, nutritional agents, nutritional oils, protease inhibitors, stimulants, or any combination thereof.

Use of the Device

The devices described herein may be used for various applications and in various environments of use. Suitable environments of use are not overly limited.

The disclosed technology further provides for the described drug delivery device where the environment of use is the mouth, ear canals, skull, gastrointestinal tract, wounds, teeth cavities, vagina, anus, stoma, eye cavities, kidneys, testicles, prostate, lungs, transplanted organs, blood vessels, the biliary tract, the urinary tract, the intestinal tract, nasal cavity, neural sheath, intervertebral regions, bone cavities, esophagus, intrauterine spaces, pancreatic and bile ducts, rectum, any location within the body just beneath an outer surface of skin, any previously intervened body spaces that have implants present, or any combination thereof or any combination thereof.

In some embodiments, the device described herein is used just beneath an outer surface of skin of the patient and/or subject. In some embodiments, the device described herein is used in the vagina.

The disclosed technology further provides the described drug delivery device where the release rate of the active agent from said drug composition to said environment of use is not controlled by the permeability of the polymeric material by the active agent or by said any fluids present in the environment of use. That is, the drug delivery rate is primarily controlled by the dissolution of the active agent into fluids present in the environment of use that come into contact with the drug composition.

In the disclosed technology, the primary means of delivery the active agent from the described drug composition to the environment of use (and/or to the subject/patient) is by dissolution of the active agent (and/or the entire drug composition) into fluids present in the environment of use. These fluids include, but are not limited to, interstitial fluids, lymph, Cerebrospinal fluid, intraocular fluids, blood, puss, saliva, ocular fluids, gastrointestinal liquids, urine, vaginal fluids, semen, etc., or any combination thereof.

In some embodiments, the fluid is blood. In some embodiments, the fluid is vaginal fluids.

The dissolution occurs with the contact of the fluids in the environment of use with the drug composition in the described device. The fluids may pass through the openings and/or passageways in the device and come into contact with the drug composition. Over the course of time, the active agent dissolves into the fluids and migrates into the environment of use. The fluids in the environment of use may move and/or flow in such a way as to cause the fluid to flow through the device, entering and exiting the device through the openings and/or passageways and in that way carrying active agent out of the device and into the environment of use.

In some embodiments, additional means of delivering the active agent to the environment of use may also be occurring. For example, the fluids present in the environment of use may permeate through the wall of the device, thus bypassing the openings and/or passageways, and coming into contact with the drug composition and picking up active agent that way. These additional means of delivering the active agent are minor in nature compared to the fluid that passes through the openings and/or passageways and comes into contact with the drug composition. By minor, in some embodiments, it is meant that less than half of the delivery of the active agent from the drug composition to the environment of use occurs by these additional means, that is at least half of the delivery of the active agent occurs my means by of the fluid passing through the openings and/or passageways and coming into contact with the drug composition. In some embodiments, this primary means of delivery accounts for at least 50%, 60%, 70%, 80%, or even 90% of the delivery of the active agent. In some embodiments, all delivery of the active agent occurs by this primary means of delivery.

The period of use for the described devices is not overly limited and is determined more by active agent and desired length of drug delivery and/or the properties of the drug composition used within the described device.

The disclosed technology also provides a means for doctors and scientist to more precisely measure the amount of drugs and/or active agents release into an environment of use by providing a device that can be accurately assayed or weighted before and after its use to calculate the amount of material that has left the device during its use.

The disclosed technology further provides for the described drug delivery device where the period of use is at least 1 month, or at least 3 months, or at least 6 months, or even a period of time needed to release substantially all of the active agent in the environment of use.

The disclosed technology further provides for the described drug delivery device where the mesh material comprises a biodegradable polymer. In such embodiments, all of the passageways may be covered by the mesh material and as the mesh material biodegrades the passageways may become more open and/or available to allow fluids present in the environment of use to pass into and out of the device, thus increasing the release rate of the active agent into the fluids and so also into the environment of use.

In this way, the described devices may be controlled release devices, and more specifically, in some embodiments the devices of the disclosed technology can react to conditions in the environment of use in such as a way as to control the rate of release active agent without the need for a power source, sensor, or other electronics or similar features. Instead, in some embodiments, the polymeric material that makes up the device and/or the mesh material may respond to one or more conditions in the environment of use in such a way as to increase or decrease the ability of fluids present in the environment of use to enter and exit the device, and so increase or decrease the rate of release of active agent into the fluid and so environment of use.

In some embodiments, the polymeric material of the device and/or the mesh material reacts to the temperature in the environment of use by swelling or contracting, thus decreasing or increasing the size of the openings and/or passageways, and so decreasing or increasing the rate of release of the active agent.

In some embodiments, the polymeric material of the device and/or the mesh material reacts to the pH of the fluids in the environment of use by swelling or contracting, thus decreasing or increasing the size of the openings and/or passageways, and so decreasing or increasing the rate of release of the active agent.

The disclosed technology further provides for the described drug delivery device where described device is made of a cross-linked polyacrylic acid polymer that is a carbomer homopolymer, a carbomer copolymer, a carbomer interpolymer, a polycarbophil or a mixture thereof.

The disclosed technology further provides for the described drug delivery device where the mesh material is made of a cross-linked polyacrylic acid polymer that is a carbomer homopolymer, a carbomer copolymer, a carbomer interpolymer, a polycarbophil or a mixture thereof.

The cross-linked polyacrylic acid polymer described above may include a cross-linked polyacrylic acid selected from one or more carbomers, one or more polycarbophils, one or more copolymers of acrylic acid and alkyl acrylates, or combinations of two or more thereof.

As used herein, the term polyacrylic acid or acrylic acid polymers is used to encompass a variety of polymers having high percentages of polymerizable monomers therein with pendant carboxylic acid groups or anhydrides of polycarboxylic acid. These compounds are described in more detail in U.S. Pat. Nos. 2,798,053; 3,915,921; 4,267,103; 5,288,814; and 5,349,030, all of which are hereby incorporated by reference in their entireties. The term polyacrylic acid is also used to include various homopolymers, copolymers, and interpolymers, wherein at least 50 or 75 mole percent of the repeating units have pendant carboxylic acid groups or anhydrides of dicarboxylic acid groups. While acrylic acid is the most common primary monomer used to form polyacrylic acid the term is not limited thereto but includes generally all alpha-beta-unsaturated monomers with carboxylic pendant groups or anhydrides of dicarboxylic acids as described in U.S. Pat. No. 5,349,030.

Suitable cross-linked polyacrylic acids include, but are not limited to polycarbophils, carbomers, Carbopol® polymers, Carbopol homopolymers, Carbopol copolymers, Carbopol interpolymers copolymers of acrylic acid and alkyl acrylates, or combinations of two or more thereof. An approved polyacrylic acid for pharmaceutical applications, described in a carbomer monograph in the U.S.P. Pharmacopeia 30 NF 25, is a polyacrylic acid crosslinked with polyalkenyl ethers.

In some embodiments, the polymer material used in the described device and/or mesh includes Carbopol 971P NF, Carbopol 71G NF, Carbopol 974P NF, or any combination thereof.

The disclosed technology further provides for the described drug delivery device where the device: (i) is not an osmotic device; (ii) is not bioresorbable; (iii) is not biodegradable; (iv) does not contain a semi-permeable membrane; (v) is not in the shape of a ring; (vi) is not significantly permeable to the active agent, any fluids present in the environment of use, or both; or (vii) all of the above.

In some embodiments, the described device is not an osmotic device. That is, the device does not make use of any osmotic processes to transfer fluid through the device nor to transfer the drug composition and/or the active agent out of the device.

In some embodiments, the described device is not bioresorbable. That is, the device does not break down and absorb in to the environment of use over time. Rather, in such embodiments, the device remains fully intact and structurally sound such that it can be removed from the environment of use as a single piece without leaving any pieces or fragments behind. In some embodiments, the device itself does not lose any mass during the period of time it is present in the environment of use, excluding the mass of the drug composition.

In some embodiments, the described device does not include a semi-permeable membrane. That is, the device is made up of a polymeric material that is not permeable to the active agent and/or the fluids present in the environment of use, or is only slightly permeable. Further, in such embodiments, the device does not include a membrane designed to allow the active agent to pass through it, nor does the device does include a membrane designed to allow the fluids present in the environment of use to pass through it. Rather, the device only includes the described openings and passageways that allow for the fluids present in the environment to enter and exit the device and come into contact with the drug composition and the active agent.

In some embodiments, the described device is not in the shape of a ring.

In some embodiments, the described device is not significantly permeable to the active agent, any fluids present in the environment of use, or both.

The disclosed technology further provides a method for using the described drug delivery device to administer a therapeutically effective amount of an active agent to an environment of use, where the drug delivery device may be any of the devices described herein, and where the method includes the steps of: (I) implanting a drug delivery device into an environment of use; (II) administering a therapeutically effective amount of an active agent from said drug composition to said environment of use during a period of use; and (III) removing said drug delivery device from said environment of use; where the drug delivery device maintains its structural integrity during said period of use.

The amount of each chemical component described is presented exclusive of any solvent which may be customarily present in the commercial material, that is, on an active chemical basis, unless otherwise indicated. However, unless otherwise indicated, each chemical or composition referred to herein should be interpreted as being a commercial grade material which may contain the isomers, by-products, derivatives, and other such materials which are normally understood to be present in the commercial grade.

It is known that some of the materials described above may interact in the final formulation, so that the components of the final formulation may be different from those that are initially added. For instance, metal ions can migrate to other acidic or anionic sites of other molecules. The products formed thereby, including the products formed upon employing the composition of the technology described herein in its intended use, may not be susceptible of easy description. Nevertheless, all such modifications and reaction products are included within the scope of the technology described herein; the technology described herein encompasses the composition prepared by admixing the components described above.

### EXAMPLES

The technology described herein may be better understood with reference to the following non-limiting examples.

A series of medical devices may be prepared made from polyethylene (PE), in multiple following shapes, with various openings, as summarized in the table below.

TABLE 1

| Exam- | Mate- | Shape of | |
|---|---|---|---|
| ple | rial | Device | Openings and Mesh |
| 1 | PE | ring | 1 opening, no mesh |
| 2 | PE | ring | 12 openings evenly spaced along outside |
| | | | of the ring, no mesh |
| 3 | PE | ring | 12 openings evenly spaced along outside |
| | | | of the ring, covered by a mesh made of a |
| | | | Carbopol ® polymer |
| 4 | PE | rectangular | 1 opening, no mesh |
| | | box | |
| 5 | PE | rectangular | 12 openings arranged in a series on |
| | | box | concentric circles, no mesh |
| 6 | PE | rectangular | 12 openings arranged in a series on |
| | | box | concentric circles, covered by a mesh |
| | | | made of a Carbopol ® polymer |
| 7 | PE | sphere | 1 opening, no mesh |
| 8 | PE | sphere | 5 openings arranged in the shape of a |
| | | | square with an opening in the center, |
| | | | no mesh |
| 9 | PE | sphere | 5 openings arranged in the shape of a |
| | | | square with an opening in the center, |
| | | | covered by a mesh made of a Carbopol ® |
| | | | polymer |
| 10 | PE | ovoid | 5 openings arranged in the shape of a |
| | | | square with an opening in the center, |
| | | | covered by a mesh made of a Carbopol ® |
| | | | polymer |

A series of medical devices may be prepared made from polypropylene (PP), in multiple following shapes, with various openings, as summarized in the table below.

TABLE 2

| Exam- | Mate- | Shape of | |
|---|---|---|---|
| ple | rial | Device | Openings and Mesh |
| 11 | PP | ring | 1 opening, no mesh |
| 12 | PP | ring | 12 openings evenly spaced along outside |
| | | | of the ring, no mesh |
| 13 | PP | ring | 12 openings evenly spaced along outside |
| | | | of the ring, covered by a mesh made of a |
| | | | Carbopol ® polymer |
| 14 | PP | rectangular | 1 opening, no mesh |
| | | box | |
| 15 | PP | rectangular | 12 openings arranged in a series on |
| | | box | concentric circles, no mesh |
| 16 | PP | rectangular | 12 openings arranged in a series on |
| | | box | concentric circles, covered by a mesh |
| | | | made of a Carbopol ® polymer |
| 17 | PP | sphere | 1 opening, no mesh |
| 18 | PP | sphere | 5 openings arranged in the shape of a |
| | | | square with an opening in the center, |
| | | | no mesh |
| 19 | PP | sphere | 5 openings arranged in the shape of a |
| | | | square with an opening in the center, |
| | | | covered by a mesh made of a Carbopol ® |
| | | | polymer |
| 20 | PP | ovoid | 5 openings arranged in the shape of a |
| | | | square with an opening in the center, |
| | | | covered by a mesh made of a Carbopol ® |
| | | | polymer |

A series of medical devices may be prepared made from polylactide (PLA), in multiple following shapes, with various openings, as summarized in the table below.

TABLE 3

| Exam- | Mate- | Shape of | |
|---|---|---|---|
| ple | rial | Device | Openings and Mesh |
| 21 | PLA | ring | 1 opening, no mesh |
| 22 | PLA | ring | 12 openings evenly spaced along outside |
| | | | of the ring, no mesh |
| 23 | PLA | ring | 12 openings evenly spaced along outside |
| | | | of the ring, covered by a mesh made of a |
| | | | Carbopol ® polymer |
| 24 | PLA | rectangular | 1 opening, no mesh |
| | | box | |
| 25 | PLA | rectangular | 12 openings arranged in a series on |
| | | box | concentric circles, no mesh |
| 26 | PLA | rectangular | 12 openings arranged in a series on |
| | | box | concentric circles, covered by a mesh |
| | | | made of a Carbopol ® polymer |
| 27 | PLA | sphere | 1 opening, no mesh |
| 28 | PLA | sphere | 5 openings arranged in the shape of a |
| | | | square with an opening in the center, |
| | | | no mesh |
| 29 | PLA | sphere | 5 openings arranged in the shape of a |
| | | | square with an opening in the center, |
| | | | covered by a mesh made of a Carbopol ® |
| | | | polymer |
| 30 | PLA | ovoid | 5 openings arranged in the shape of a |
| | | | square with an opening in the center, |
| | | | covered by a mesh made of a Carbopol ® |
| | | | polymer |

Each of the devices above may include the following drug compositions: Drug Composition 1: a mixture of yellow carnauba which is melted along with an active pharmaceutical ingredient to form a single phase, and emulsified into a solution mixture of 1% Brij700 non-ionic emulsifier and 1% chitosan in 1% acetic acid (to dissolve the chitosan). After emulsification the dispersion is cooled to form a drug composition.

Each of the devices may then be inserted into an environment of use, where examples 1 to 10 may be inserted into a vagina, examples 11 to 20 may be inserted just below the skin of the forearm, and examples 21 to 30 may be inserted just below the skin of the abdomen. Each of the devices may be left in the environment of use for a period of 1 month, 3 months, or 6 six months, and then removed from the subject. During which time the active pharmaceutical ingredient will have released into the subject.

Each of the documents referred to above is incorporated herein by reference, including any prior applications, whether or not specifically listed above, from which priority is claimed. The mention of any document is not an admission that such document qualifies as prior art or constitutes the general knowledge of the skilled person in any jurisdiction. Except in the Examples, or where otherwise explicitly indicated, all numerical quantities in this description specifying amounts of materials, reaction conditions, molecular weights, number of carbon atoms, and the like, are to be understood as modified by the word “about.” It is to be understood that the upper and lower amount, range, and ratio limits set forth herein may be independently combined. Similarly, the ranges and amounts for each element of the technology described herein can be used together with ranges or amounts for any of the other elements.

As described hereinafter the molecular weight of the materials described above have been determined using known methods, such as GPC analysis using polystyrene standards. Methods for determining molecular weights of polymers are well known. The methods are described for instance: (i) P. J. Flory, “Principles of star polymer Chemistry”, Cornell University Press 91953), Chapter VII, pp 266-315; or (ii) “Macromolecules, an Introduction to star polymer Science”, F. A. Bovey and F. H. Winslow, Editors, Academic Press (1979), pp 296-312. As used herein the weight average and number weight average molecular weights of the materials described are obtained by integrating the area under the peak corresponding to the material of interest, excluding peaks associated with diluents, impurities, uncoupled star polymer chains and other additives.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the basic and novel characteristics of the composition or method under consideration. That is “consisting essentially of” permits the inclusion of substances that do not materially affect the basic and novel characteristics of the composition under consideration.

While certain representative embodiments and details have been shown for the purpose of illustrating the subject technology described herein, it will be apparent to those skilled in this art that various changes and modifications can be made therein without departing from the scope of the subject invention. In this regard, the scope of the technology described herein is to be limited only by the following claims.

1. A drug delivery device for administering a therapeutically effective amount of an active agent to an environment of use, the device comprising:
(a) a wall comprising at least in part a polymeric composition, a metallic material, or a combination thereof;
which wall surrounds and forms;
(b) a compartment;
(c) a drug composition in the compartment, said drug composition comprising said active agent, wherein said drug composition is in a formed shape that fits within said compartment;
(d) at least one passageway in the wall communicating with the compartment and the exterior of the device for releasing the active agent from said drug composition to said environment of use; and
(e) a mesh material positioned on the wall of the device such that it covers one or more of the passageways.
wherein said device (i) can be implanted in the environment of use, (ii) is able to maintain its structural integrity in the environment of use for a period of use such that a therapeutically effective amount of the active agent releases from said drug composition into said environment of use, and (iii) can then be removed from the environment of use, or left in place to eventually degrade after the active agent has been released.

2. The drug delivery device of claim 1 wherein each passageway is of a shape or size that prevents the entirety of said drug composition from passing through the passageway and out of the compartment.

3. The drug delivery device of claim 1 wherein the release rate of the active agent from said drug composition to said environment of use is not controlled by the permeability of said polymeric material by said active agent or by said any fluids present in the environment of use.

4. The drug delivery device of claim 1 wherein the period of use is at least 1 month.

5. The drug delivery device of claim 1 where said wall is free of metallic materials and where said polymeric material comprises:
(a) a biodegradable polymer;
(b) a non-biodegradable polymer;
(c) any combination thereof.

6. The drug delivery device of claim 1 where said active agent comprises one or more analgesics, anti-anginal agents, anti-arrhythmic agents, anti-angiogenic agents, antibacterial agents, anti-benign prostate hypertrophy agents, anti-coagulants, anti-depressants, anti-diabetic agents, anti-epileptic agents, anti-fungal agents, anti-gout agents, anti-hypertensive agents, anti-inflammatory agents, anti-malarial agents, anti-migraine agents, anti-muscarinic agents, anti-neoplastic agents, anti-obesity agents, anti-osteoporosis agents, anti-parkinsonian agents, anti-protozoal agents, anti-thyroid agents, anti-urinary incontinence agents, anti-viral agents, anxiolytics, beta-blockers, cardiac inotropic agents, cognition enhancers, corticosteroids, COX-2 inhibitors, diuretics, erectile dysfunction improvement agents, essential fatty acids, gastrointestinal agents, histamine receptor antagonists, hormones, immunosuppressants, keratolyptics, leukotriene antagonists, lipid regulating agents, macrolides, muscle relaxants, non-essential fatty acids, nutritional agents, nutritional oils, protease inhibitors, stimulants, or any combination thereof.

7. The drug delivery device of claim 1 where said environment of use is the mouth, ear canals, skull, gastrointestinal tract, wounds, teeth cavities, vagina, anus, stoma, eye cavities, kidneys, testicles, prostate, lungs, transplanted organs, blood vessels, the biliary tract, the urinary tract, the intestinal tract, nasal cavity, neural sheath, intervertebral regions, bone cavities, esophagus, intrauterine spaces, pancreatic and bile ducts, rectum, any location within the body just beneath an outer surface of skin, any previously intervened body spaces that have implants present, or any combination thereof or any combination thereof.

8. The drug delivery device of claim 1 where said device comprises a series of passageways arranged in a pattern on at least one surface of said device.

9. The drug delivery device of claim 1 where said device is made by means of 3D printing, and wherein said device is printed as a single continuous piece where said drug composition is inserted into said compartment with the device while the device is being printed.

10. The drug delivery device of claim 1 wherein said mesh material covers all of the passageways of the device, and wherein said mesh material comprises a polymeric material that swells when in contact with an environmental agent that may be present in the environment of use, such that when the environmental agent is present, the mesh material swells and substantially blocks the passageways of said device, restricting the release of the active agent from the drug composition to the environment of use.

11. The drug delivery device of claim 1 wherein the mesh material comprises a biodegradable polymer;
wherein said biodegradable polymer comprises a cross-linked polyacrylic acid polymer that is a carbomer homopolymer, carbomer copolymer, carbomer interpolymer, polycarbophil or a mixture thereof.

12. The drug delivery device of claim 1 wherein the drug delivery device is coated, treated, or both with a material to minimize scaring or encapsulation of said device by the environment of use.

13. The drug delivery device of claim 1 wherein said device: (i) is not an osmotic device; (ii) is not bioresorbable; (iii) is not biodegradeable; (iv) does not contain a semi-permeable membrane; (v) is not in the shape of a ring; (vi) is not significantly permeable to the active agent, any fluids present in the environment of use, or both; or (vii) all of the above.

14. A method of using a drug delivery device to administer a therapeutically effective amount of an active agent to an environment of use said drug delivery device comprising:
(a) a wall comprising at least in part a polymeric composition, which wall surrounds and forms;
(b) a compartment;
(c) a drug composition in the compartment, said drug composition comprising said active agent, wherein said drug composition is in a formed shape that fits within said compartment;
(d) at least one passageway in the wall communicating with the compartment and the exterior of the device for delivering the active agent from said drug composition to said environment of use, where each passageway is of a shape or size that prevents said drug composition from passing through the passageway and out of the compartment; and
(e) a mesh material positioned on the wall of the device such that it covers one or more of the passageways;
said method comprising the steps of:
(I) implanting a drug delivery device into an environment of use;
(II) administering a therapeutically effective amount of an active agent from said drug composition to said environment of use during a period of use;
(III) removing said drug delivery device from said environment of use;
wherein said drug delivery device maintains its structural integrity during said period of use.
